(19) United States
(12) Patent Application Publication
(10) Pub. No.: US 2025/0114282 A1
(43) Pub. Date: Apr. 10, 2025

(54) Dental materials and methods of making and using thereof

(71) Applicant: Chong HUANG, San Ramon, CA (US)

(72) Inventor: Chong HUANG, San Ramon, CA (US)

(73) Assignee: Chong HUANG, San Ramon, CA (US)

(21) Appl. No.: 18/484,442

(22) Filed: Oct. 10, 2023

(51) Int. Cl. A61K 6/891; A61C 5/00; A61K 6/62; A61K 6/831; C08F 220/28; C08F 220/32; C08F 222/10; C08F 222/22; C08K 3/04

(52) U.S. Cl. CPC A61K 6/891; A61C 5/00; A61K 6/62; A61K 6/831; C08F 220/281; C08F 220/325; C08F 222/1025; C08F 222/22; C08K 3/04; C08F 2800/20

(57) ABSTRACT

A novel diamond Resin Composite (DRC) includes 30-60 wt % diamond particles and a multi-monomer resin blend, enhanced with select photoinitiators and coinitiators. The diamond particles can be synthetic, acquired through high-pressure high-temperature (HPHT), or chemical vapor deposition (CVD) methods or hot-filament chemical vapor deposition (HFCVD) or DC plasma enhanced chemical vapor deposition (DC-PECVD) methods. The DRC contains fluorine doping of diamonds, achieved via reactive gas precursors or fluorine-rich plasma. Supplementary agents enrich the composite's versatility. The diamonds within the DRC are diverse in size, shape, and surface treatments, notably featuring terminations by hydrogen, oxygen, or fluorine. In dental restorations, diamonds are combined with a light-sensitive resin, and the mixture undergoes light-induced photopolymerization.

### BACKGROUND

In recent years, the rapid advancement of biomaterials has highlighted diamonds and their composites as significant areas of research.

### SUMMARY

The present disclosure relates to the field of dental composite materials. More specifically, the present disclosure relates to diamond composite materials and applications for dental restoration using synthetic diamond in combined with composite resins to effectuate desired results.

In the realm of dental restorations, the quest for materials that are durable, wear-resistant, chemically stable, and aesthetically appealing has been ongoing. Addressing this challenge, the Diamond Resin Composite (DRC) is provided in some embodiments of the present disclosure. The DRC includes synthetic diamond particles of various weight percentages, which are obtained via methods such as HPHT synthesis and various CVD techniques. These particles undergo notable modifications to enhance their properties. These modified diamonds are blended with a multi-monomer resin mixture forming the Diamond Resin Composite (DRC) which is fortified optionally with supplementary compounds. The doping of diamond particles is a transformative step that further enhances the composites. Beyond the material composition, the DRC's application to dental restoration is facilitated through its compatibility with light-induced photopolymerization, ensuring a reliable bond with natural teeth. Distinct from previous composites, the DRC's integration of modified diamonds presents a game-changing advancement with unparalleled strength, optimal biocompatibility, and an aesthetic mirroring natural enamel for dental industry.

A diamond resin composite (DRC) includes diamond particles, a multi-monomer resin mixture, wherein the diamond particles are embedded within the multi-monomer resin mixture, photoinitiator and coinitiator.

The diamond particles in the diamond resin composite (DRC) are made from bulk diamond, wherein the diamond is of synthetic origin.

The dental diamond resin composite in the diamond resin composite (DRC) includes a resin matrix, wherein the matrix selectively includes one or more of the following components: Bisphenol A-glycidyl methacrylate (Bis-GMA) in a concentration ranging from 10% to 60% by weight; Triethylene glycol dimethacrylate (TEGDMA) in a concentration ranging from 10% to 40% by weight; Urethane dimethacrylate (UDMA) in a concentration ranging from 10% to 40% by weight; Hydroxyethyl methacrylate (HEMA) in a concentration ranging from 5% to 15% by weight, and; Ethoxylated Bisphenol A dimethacrylate (Bis-EMA) in a concentration ranging from 10% to 50% by weight.

The resin matrix in the dental diamond resin composite primarily consists of Bis-GMA.

Optionally, the resin matrix in the dental diamond resin composite primarily consists of TEGDMA.

Optionally, the resin matrix in the dental diamond resin composite primarily consists of UDMA.

Optionally, the resin matrix in the dental diamond resin composite primarily consists of HEMA.

Optionally, the resin matrix in the dental diamond resin composite primarily consists of Bis-EMA.

The resin matrix in the dental diamond resin composite of any preceding options includes a blend of at least two of the components as mentioned above.

A method for preparing a dental diamond resin composite is provided, wherein the method includes mixing a resin blend selectively including one or more components as defined above.

The resin blend in the diamond resin composite (DRC) further includes 5-25 wt % ethylene glycol dimethacrylate.

The diamond resin composite in a dental resin composite (DRC) includes a photopolymerization capability and at least one photoinitiator wherein the photoinitiator initiates polymerization of resin monomers upon exposure to light.

The photoinitiator in the dental resin composite (DRC) includes camphorquinone (CQ).

Upon exposure to visible light, the CQ in the dental resin composite (DRC) absorbs the light energy and transitions to an excited state thereby inducing polymerization.

The photoinitiator in the dental resin composite (DRC) includes at least one of 9-(2,4,4,6-trimethylbenzoyl)-9-oxytho-9-phospha-fluoren (TMBOPF) or 9-(p-toluyl)-9-oxytho-9-phosphafuluorene (TOPF).

The concentration of the photoinitiator in the dental resin composite (DRC) is between about 0.01-0.5 wt %.

The photoinitiator in the dental resin composite (DRC) is selected from the group consisting of camphorquinone (CQ), 9-(2,4,4,6-trimethylbenzoyl)-9-oxytho-9-phospha-fluoren (TMBOPF), and 9-(p-toluyl)-9-oxytho-9-phosphafuluorene (TOPF).

The dental resin composite (DRC) further includes Phenylpropanedione (PPD) conjoined with CQ, wherein the combination amplifies polymerization efficiency.

The dental resin composite (DRC) further includes Lucirin TPO (LAP), wherein the LAP augments the curing process.

The dental resin composite (DRC) further including Ivocerin (BAPO).

A method of preparing a dental restoration is provided in the present application, which includes applying the DRC to a dental site and exposing the DRC to light to induce polymerization, wherein the light has a wavelength of approximately 468 nm.

The dental resin composite in the dental resin composite (DRC) includes a polymerizable resin matrix, a photoinitiator; and a co-initiator, wherein the co-initiator is present in a concentration ranging from about 0.01 to 0.5 wt %.

The co-initiator in the dental resin composite is ethyl N,N-dimethyl-4-aminobenzoate (EDMAB).

Optionally, the co-initiator in the dental resin composite is 1,3-Diethyl-2-thiobarbituric acid.

The dental resin composite further includes at least one additional co-initiator selected from the group consisting of Ethyl 4-(dimethylamino)benzoate (EDAB),N,N-dimethyl-p-toluidine (DMPT), N,N-dihydroxyethyl-p-toluidine (DHEPT), Triethanolamine (TEA), and Cyanoethyl methylaniline (CEMA).

The diamond materials in the diamond resin composite (DRC) are prepared using high-pressure high-temperature (HPHT) synthesis or chemical vapor deposition (CVD) methods or Hot-filament chemical vapor deposition (HFCVD) or DC plasma enhanced chemical vapor deposition (DC-PECVD) methods.

The synthetic Diamond in the synthetic diamond is produced using a high-pressure, high-temperature (HPHT) process, wherein the diamond includes nickel (Ni) as an element with a concentration not exceeding 200 parts per million (ppm).

In some embodiments, the synthetic Diamond in the synthetic diamond is produced using a high-pressure, high-temperature (HPHT) process, wherein the diamond includes nitrogen (N) as an element with a concentration not exceeding 100 parts per million (ppm).

In some embodiments, the synthetic Diamond in the synthetic diamond is produced using a high-pressure, high-temperature (HPHT) process, wherein the diamond includes iron (Fe) as an element with a concentration not exceeding 200 parts per million (ppm).

In some embodiments, the synthetic Diamond in the synthetic diamond is produced using a high-pressure, high-temperature (HPHT) process, wherein the diamond includes cobalt (Co) as an element with a concentration not exceeding 200 parts per million (ppm).

In some embodiments, the synthetic Diamond in the synthetic diamond is produced using a high-pressure, high-temperature (HPHT) process, wherein the diamond includes magnesium (Mg) as an element with a concentration not exceeding 100 parts per million (ppm).

Optionally, the synthetic Diamond in the synthetic diamond is produced using a Microwave plasma-assisted chemical vapor deposition (MPCVD) process, wherein the diamond includes nitrogen as an element with a concentration not exceeding 200 parts per million (ppm).

In some embodiments, the synthetic Diamond in the synthetic diamond is produced using a Microwave plasma-assisted chemical vapor deposition (MPCVD) process, wherein the diamond includes boron as an element with a concentration not exceeding 300 parts per million (ppm).

In some embodiments, the synthetic Diamond in the synthetic diamond is produced using a Microwave plasma-assisted chemical vapor deposition (MPCVD) process, wherein the diamond includes silicon (Si) as an element with a concentration not exceeding 1 part per thousand (ppt).

The synthetic Diamond in the synthetic diamond is produced using a Microwave plasma-assisted chemical vapor deposition (MPCVD) process, wherein the MPCVD process including the introduction of a gas mixture into the deposition chamber, the gas mixture consisting of a carbon source selected from the group consisting of methane (CH4), carbon dioxide (CO2), and combinations thereof, and hydrogen (H2) with hydrogen ratio in gas mixture no less than 80 vol. %.

In some embodiments, the synthetic Diamond in the synthetic diamond is produced using a Microwave plasma-assisted chemical vapor deposition (MPCVD) process, wherein the MPCVD process including the control of the diamond growth temperature, wherein the growth temperature ranging from 300 degrees Celsius to 1400 degrees Celsius.

In some embodiments, the synthetic Diamond in the synthetic diamond is produced using a Microwave plasma-assisted chemical vapor deposition (MPCVD) process, wherein the MPCVD process employing a microwave generator to create a plasma within the chemical vapor deposition reactor chamber, and the plasma at a pressure ranging from 1×10−7 Torr to 100 Torr.

Optionally, the synthetic Diamond in the synthetic diamond is produced using a Hot-filament chemical vapor deposition (HFCVD) process, wherein the HFCVD process including the introduction of a gas mixture into the deposition chamber, wherein the gas mixture consisting of a carbon source selected from the group consisting of methane (CH4), carbon dioxide (CO2), carbon monoxide (CO) and combinations thereof, and hydrogen (H2) with hydrogen ratio in gas mixture no less than 80 vol. %.

In some embodiments, the synthetic Diamond in the synthetic diamond is produced using a Hot-filament Chemical Vapor Deposition (HFCVD) process, wherein the HFCVD process including heating a filament to a temperature in the range of 1000 degrees Celsius to 2400 degrees Celsius; and depositing diamond materials within the reactor chamber under controlled temperature conditions.

In some embodiments, the synthetic Diamond in the synthetic diamond is produced using a Hot-filament Chemical Vapor Deposition (HFCVD) process, the process including:
employing a chemical vapor deposition reactor chamber;
maintaining a pressure within the reactor chamber in the range of 1×10−4 Torr to 760 Torr; and
depositing the diamond film onto a substrate or growing bulk diamond materials within the reactor chamber under controlled growth pressure conditions.

Optionally, the synthetic Diamond in the synthetic diamond is produced using a DC plasma enhanced chemical vapor deposition (DC-PECVD) process, the process including:
providing a deposition chamber;
introducing a gas mixture into the deposition chamber, the gas mixture consisting of a carbon source selected from the group consisting of methane (CH4), carbon dioxide (CO2), carbon monoxide (CO), acetylene (C2H2) and combinations thereof, and hydrogen (H2) with a hydrogen ratio in gas mixture of no less than 50 vol. %; and
growing diamond materials within the reactor chamber under controlled conditions.

In some embodiments, the synthetic Diamond in the synthetic diamond is produced using a DC plasma enhanced chemical vapor deposition (DC-PECVD) process, the process including:
providing a deposition chamber;
growing diamond material with the substrate temperature within the deposition chamber in a range between 20 degrees Celsius and 1500 degrees Celsius; and
growing bulk diamond materials within the reactor chamber under controlled temperature conditions.

In some embodiments, the synthetic Diamond in the synthetic diamond is produced using a DC plasma enhanced chemical vapor deposition (DC-PECVD) process, the process including:
employing a plasma enhanced chemical vapor deposition reactor chamber;
maintaining a growth pressure within the reactor chamber in the range of 1×10−3 Torr to 760 Torr; and
growing bulk diamond materials within the reactor chamber under controlled growth pressure conditions.

A diamond resin composite (DRC) includes: diamond particles, wherein the particles are at a concentration of 1-60 wt % and are of synthetic diamond origin; a multi-monomer resin mixture, wherein the diamond particles are embedded in the multi-monomer resin mixture and wherein the resin blend includes 30-40 wt % bisphenol A diglycidyl ether dimethacrylate or Bisphenol A-glycidyl methacrylate.; 0.01-0.5 wt % camphorquinone (CQ), or 9-(2,4,4,6-trimethylbenzoyl)-9-oxytho-9-phospha-fluoren (TMBOPF) or 9-(p-toluyl)-9-oxytho-9-phosphafuluorene (TOPF) as the photoinitiator; 0.01-0.5 wt % ethyl N,N-dimethyl-4-aminobenzoate (EDMAB) or 1,3-Diethyl-2-thiobarbituric acid as the coinitiator.

A method is provided, including: forming a doped region of a diamond substrate by doping a surface of the diamond substrate with dopants; driving the dopants into the diamond substrate by annealing the diamond substrate; controlling doping profile of the doped region by repeating doping and annealing the diamond substrate.

The diamond particles in the diamond particles include fluorine element.

A doping method wherein fluorine doping of diamond particles is achieved by introducing fluorine-containing reactive gas precursor molecules into the diamond synthesis process, providing a method for integrating fluorine into the diamond substrate.

Optionally, a doping method wherein fluorine doping of diamond particles is achieved by incorporation of the fluorine element into the diamond substrate occurs via the presence of a fluorine-containing plasma during the diamond growth process.

The diamond particles in the diamond particles have size dimensions in a range of 10 nm-100 μm.

A method for producing diamond particles from bulk diamond includes: introducing the bulk diamond into a ball mill equipped with milling media selected from a group consisting of tungsten carbide and steel; operating the ball mill at a predetermined rotation speed for a specified duration to energetically grind and fracture the bulk diamond, thereby reducing its size to a particle range; and subsequently separating the diamond particles from the milling media and purifying the obtained diamond particles.

The diamond resin composite (DRC) in a diamond resin composite (DRC) optionally includes one or more of the following:
a) Fluoride-Releasing Resins in an amount ranging from 1% to 20% by weight;
b) Quaternary Ammonium Compounds (QACs) in an amount ranging from 0.5% to 5% by weight;
c) Silver Particles in an amount ranging from 0.05% to 2% by weight;
d) Zinc Oxide Particles in an amount ranging from 1% to 10% by weight;
e) Chlorhexidine in an amount ranging from 0.5% to 5% by weight; and
f) Bioactive Glass in an amount ranging from 5% to 15% by weight.

The diamond particles have a non-spherical shape characterized by a sphericity ranging between 0.07 and 0.97.

The diamond particles are surface treated by plasma prior to mixing with other constituent materials to form diamond resin composite (DRC) or alternatively remain untreated.

The surface of the diamond particles is hydrogen-terminated or alternatively remains untreated.

Optionally, the surface of the diamond particles is Oxygen-terminated or alternatively remain untreated.

Optionally, the surface of the diamond particles is fluorine-terminated or alternatively remain untreated.

A dental restoration method is provided, including:
a) providing the diamond resin composite (DRC);
b) applying the diamond resin composite (DRC) onto a surface of a dental structure in need of restoration; and
c) illuminating the diamond resin composite (DRC) with a light source of an appropriate wavelength to induce photopolymerization of the diamond resin composite (DRC), thereby restoring the dental structure.

In some embodiments, the diamond resin composite (DRC) in the dental restoration method is applied as a direct filling into cavities of both anterior and posterior teeth.

In some embodiments, the diamond resin composite (DRC) in the dental restoration method is custom formed to fit dental cavities and cemented into place as inlays and onlays.

In some embodiments, the diamond resin composite (DRC) in the dental restoration method is applied for minor cosmetic corrections through dental bonding.

In some embodiments, the diamond resin composite (DRC) in the dental restoration method is used to create veneers, forming a thin shell bonded to the front side of teeth.

In some embodiments, the diamond resin composite (DRC) in the dental restoration method is used for core build-ups, reconstructing a tooth's core before crown placement.

In some embodiments, the diamond resin composite (DRC) in the dental restoration method is employed in splinting, connecting unstable teeth to neighboring stable teeth.

In some embodiments, the diamond resin composite (DRC) in the dental restoration method is used for cusp protection, reinforcing a fragile tooth.

In some embodiments, the diamond resin composite (DRC) in the dental restoration method is applied as sealants on the occlusal surfaces of molars and premolars.

In some embodiments, the diamond resin composite (DRC) in the dental restoration method is utilized in orthodontic treatments, bonding brackets to teeth.

In some embodiments, the diamond resin composite (DRC) in the dental restoration method is employed for root surface caries restorations, treating decay on root surfaces.

In some embodiments, the diamond resin composite (DRC) in the dental restoration method is applied in pediatric dentistry for the treatment of cavities, fractures, or developmental conditions in primary teeth.

In some embodiments, the diamond resin composite (DRC) in the dental restoration method is used for the closure of diastema, sealing gaps between teeth.

In some embodiments, the diamond resin composite (DRC) in the dental restoration method is applied for the treatment of tooth wear and erosion, restoring teeth affected by bruxism or acid erosion.

In some embodiments, the diamond resin composite (DRC) in the dental restoration method is used to address peg-shaped lateral incisors, reshaping, or enlarging these teeth.

In some embodiments, the diamond resin composite (DRC) in the dental restoration method is applied as a post and core in root canal-treated teeth before crown placement.

In some embodiments, the diamond resin composite (DRC) in the dental restoration method is utilized for Class IV restorations, addressing front teeth that are fractured or chipped.

In some embodiments, the diamond resin composite (DRC) in the dental restoration method is used for Class V restorations, addressing decay or abrasions at the gum line.

In some embodiments, the diamond resin composite (DRC) in the dental restoration method is used in the repair of dental prosthetics such as mending chips on a denture tooth.

In some embodiments, the diamond resin composite (DRC) in the dental restoration method is applied as a direct pulp capping layer over an exposed pulp.

In some embodiments, the diamond resin composite (DRC) in the dental restoration method is utilized in the reattachment of fractured tooth fragments.

In some embodiments, the diamond resin composite (DRC) in the dental restoration method is used to reshape over-retained primary teeth, guiding the eruption of permanent successors.

In some embodiments, the diamond resin composite (DRC) in the dental restoration method is employed in smile makeovers, reshaping teeth for a more aesthetic appearance.

In some embodiments, the diamond resin composite (DRC) in the dental restoration method is used for interproximal reduction in orthodontics, following the removal of a thin layer of tooth structure.

In some embodiments, the diamond resin composite (DRC) in the dental restoration method is applied for the repair of acid erosion/abrasion, addressing tooth structure loss.

A method for dental restoration including:
obtaining dental measurements from a patient;
utilizing a resin composite as defined previously; and
fabricating a custom dental component based on the dental measurements using 3D printing technology wherein the resin composite is used as a primary material.

The diamond in the synthetic diamond has a refractive index of at least 2.4 but smaller than 2.5.

The size distribution of diamond particles dispersed within the diamond resin composition, wherein the amount of diamond particles with a z-score of particle size smaller than −8 or larger than 8, relative to the mean particle size, constitutes less than 1 wt. % of the total composition weight.

The mean size of the diamond particles is in the range of 10 nm to 90 micrometers.

Optionally, the mean size of the diamond particles is in the range of 50 nm to 50 micrometers.

Optionally, the mean size of the diamond particles is in the range of 100 nm to 10 micrometers.

### BRIEF DESCRIPTION OF THE DRAWINGS

The following drawings are provided to help illustrate various features of non-limiting examples of the disclosure and are not intended to limit the scope of the disclosure or exclude alternative implementations.

FIG. 1 shows Transmission Electron Microscopy (TEM) and Selected Electron Diffraction (SAD) results of synthetic diamond formed by MPCVD. (a) High magnification Transmission electron microscopy of diamond made by MPCVD. Scale bar, 5 nm; and (b) corresponding selected area diffraction pattern.

FIG. 2 is a diagram illustrating an example of a Raman spectrum of the synthetic diamond.

FIG. 3. Shows an example of UV-Vis spectrum for transparent synthetic diamond materials in the visible range

FIG. 4 shows an example of XRD pattern for transparent synthetic diamond materials.

FIG. 5A shows an array of synthetic diamond particles, each with their distinct shape. From classic octahedral and dodecahedral forms to more irregular and fragmented shapes that may arise from mechanical processing or unique growth conditions.

FIG. 5B shows the optical microscope image of the diamond particles.

FIG. 5C shows the scanning electron microscope image of diamond particles.

FIG. 6 shows an example of an MPCVD device used for making synthetic diamonds.

FIG. 7A shows an example of variation of Secondary ionic strength with depth in SIMS test of synthetic diamond.

FIG. 7B shows the impurity levels of the example of FIG. 7A.

FIG. 8A shows an example of damaged inlay, onlay and crown before restoration.

FIG. 8B shows an example of inlay, onlay and crown after using DRC for restoration.

FIG. 9 shows an example of using DRC for resin-bonded bridges.

FIG. 10 shows an example of using DRC for dental implant.

FIG. 11 shows an example of using DRC for veneer repair.

FIG. 12 shows an example of using DRC for combined maxillary prostheses.

FIG. 13 shows the steps of applying DRC for dental restoration.

### DETAILED DESCRIPTION

The inventor of the present disclosure has recognized that dental restoration and material science consistently strive for materials that are durable, biocompatible, and can enhance the natural structure of teeth. While traditional dental restoratives met basic needs, they often fell short in aesthetics, strength, and durability. Emerging research suggests that diamond, due to its inherent strength, chemical stability, aesthetic appeal, wear resistance and biocompatibility, is becoming a frontrunner for innovative dental applications. As research progresses, the applications and potential benefits of diamond and diamond composites in dental restoration are paving the way for potential advancements that could revolutionize dental care. Some embodiments of the present disclosure can bridge the gap between the inherent strength of diamonds and the adaptability of resins, ensuring aesthetics, strength, and durability.

Diamond is a highly biocompatible material that has recently emerged as a promising candidate for dental applications. Its exceptional properties such as resistance to corrosion, inertness to chemical reactions and temperature changes, high hardness, high thermal conductivity, and transparency make it ideal for use in dental implants, crowns, and other dental components.

The oral cavity is a complex environment that is constantly exposed to a variety of factors that can affect its pH level. Normally, the pH of the oral cavity is around 7.0, which is considered neutral. However, this value may change due to extrinsic sources such as diet, medication, or certain dental procedures. It is important to note that the pH of the oral cavity plays a crucial role in determining the durability of dental materials. This is because the slightly acidic environment of the oral cavity can cause corrosion, degradation, and wear of dental materials over time.

One of the most significant advantages of diamond is its extremely high corrosive resistance. Unlike metals, which can corrode over time due to exposure to the acidic environment of the oral cavity, diamond is virtually immune to such degradation. Additionally, diamond is inert to chemical reactions and temperature changes, which are two common factors that cause dimensional changes in dental materials. This makes it highly durable and long-lasting in the oral environment.

Diamond's hardness and tribological properties are another important factor that make it an attractive material for dental applications. It is one of the hardest materials known, making it resistant to dimensional changes, scratches, and indentations. This property helps maintain the structural integrity of dental restorations and implants.

Furthermore, diamonds have high thermal conductivity, which helps to maintain a balance of heat within the mouth. This is particularly important for dental restorations that come into direct contact with the oral environment. Diamonds are also a transparent material that can change color through doping. This allows for customization to match the specific dental application and provides an aesthetically pleasing appearance.

Diamond's exceptional properties make it an attractive material for use in dental restorations, implants, and other applications where biocompatibility, durability, and performance are critical factors. As research continues to uncover new ways to utilize diamond in dental applications, it has the potential to become a game-changing material in the field of dentistry. This led to the exploration and development of composites that integrate the superior properties of diamonds with the flexibility and versatility of resin mixtures.

Some embodiments of the present disclosure provide a diamond resin composite, herein referred to as the DRC. The DRC is specially formulated with diamond particles. These diamond particles are evenly dispersed within a matrix formed by a multi-monomer resin mixture. In certain preferred embodiments, the diamond particles utilized in the DRC are of synthetic origin. The advantage of using synthetic diamond particles lies in their consistent quality, reduced impurities, and predictable performance characteristics.

Some embodiments of the present disclosure provide a diamond resin composite (DRC) that significantly advances the state of the art. The composite, comprising 30-60 wt % diamond particles, is an innovative blend, leveraging both synthetic diamond particles produced through sophisticated processes such as high-pressure high-temperature (HPHT) synthesis and three different chemical vapor deposition (CVD) techniques, and a multi-monomer resin mixture. This resin mixture is uniquely formulated ensuring optimal bonding and stability. Moreover, the incorporation of tailored photoinitiators and coinitiators ensures controlled, light-induced photopolymerization.

Mechanical downsizing of bulk diamond materials, synthesized by HPHT or CVD techniques, provides an effective approach to obtain particles in the micro and nano scales tailored for dental applications. By harnessing a high-energy ball mill and optimizing parameters such as milling speed, duration, and ball-to-powder ratio, precise control over particle size and shape can be achieved. Such meticulous optimization ensures that the resultant diamond particles are ideally suited for enhancing the performance of dental restorative materials, offering superior wear resistance, and promoting optimal aesthetics in dental prosthetics and restorations.

In addition to mechanical milling, laser ablation offers another avenue for the precision downsizing of diamond materials. This method utilizes focused laser beams to vaporize the material in a controlled manner, producing particles that can be fine-tuned down to the nanometer scale. The absence of physical grinding or mechanical stress during laser ablation means minimal introduction of defects, preserving the intrinsic properties of the diamond. Furthermore, techniques such as ion beam milling and electrochemical etching expand the toolkit for achieving nanoscale dimensions. Each method comes with its unique advantages and considerations, allowing for a multi-faceted approach to crafting diamond particles that best meet the stringent requirements of dental applications.

Some embodiments of the present disclosure provide a composition containing various constituents, with a particular emphasis on diamond particles. The specific weight percentage of diamond particles, relative to the overall weight of the composition, is precisely delineated due to its significant influence on the composition's properties and performance.

For the lower concentration limits, the composition may incorporate diamond particles at a minimum of 1 wt. %. In some embodiments, this concentration can be elevated to at least 10 wt. %, and in certain formulations, it might reach or even exceed 30 wt. %.

Conversely, for upper concentration limits, it's imperative that diamond particles do not constitute more than 60 wt. % of the composition. In alternative embodiments, the upper concentration limit might be further restricted to 55 wt. % or even as stringent as 50 wt. %.

The permissible range for diamond particles' concentration in the composition thus can extend from as low as 1 wt. %, spanning upwards to 60 wt. %. In specific formulations, this range might be narrowed, for instance, starting from 10 wt. % and reaching up to 55 wt. % or commencing at 30 wt. % and culminating at 50 wt. %.

Throughout this description, it is to be understood that the term “weight percentage” explicitly denotes the weight of diamond particles in comparison to the total weight of the composition. This delineation ensures that the formulation can be precisely tailored to optimize the desired properties and performance in various applications.

Some embodiments of the present disclosure provide compositions characterized by specific diamond particle size distributions, with the bimodal size distribution serving as a notable example for maximizing packing density. In bimodal size distribution, the relationship between the mean sizes of larger and smaller particles becomes critical. An optimal balance between these mean sizes is achieved when a particle size ratio, derived from the comparison of the larger mean particle size to the smaller mean particle size, falls within a range of 2 to 20. This ratio is deemed optimal for the composition, and by adhering to it, the composition showcases enhanced packing properties.

Specifically, smaller particles, following this ratio, are tailored to efficiently occupy the voids or spaces typically left by the larger particles, leading to a cohesive and dense packing. The particle size ratio holds significance not just for bimodal distributions but could provide insights for other size distributions as well, by extending the principles laid down by this relationship.

In some embodiments of the present disclosure, the inclusion of smaller particles in a composition, while advantageous in void filling, can present challenges. Notably, when extremely fine particles are introduced in significant amounts, there can be an unintended change in overall resin density. This variance can be attributed primarily to increased interparticle forces associated with smaller particle sizes. Additionally, the presence of these fine particles can heighten the risk of air entrapment in the composition, further impeding optimal packing. As such, while smaller particles offer benefits, their potential drawbacks, especially when present in large amounts, are carefully considered in composition design.

Some embodiments of the present disclosure provide specific size distributions of diamond particles within a composition and the utilization of statistical measures, specifically the z-score, to quantify and ensure the consistency and uniformity of the distribution. For clarity, the z-score is a statistical metric used to determine how many standard deviations a particular data point, or in this context, a diamond particle, deviates from the mean size of the particles in the distribution.

In one embodiments, diamond particles are uniformly dispersed throughout the composition. A characteristic of interest in this embodiment is the z-score associated with the size of these diamond particles. Specifically, the composition ensures that diamond particles possessing a z-score smaller than −8 or larger than 8, when compared relative to the mean particle size, constitute less than 1 wt % of the total weight of the composition. This constraint ensures that extreme outliers, in terms of particle size, are minimized in the composition, leading to more predictable and consistent material properties.

Some embodiments of the present disclosure provide the enhancement of diamond through methods of doping and surface alteration. Doping not only enhances the properties of diamond particles but also introduces additional functionalities. These diamond particles are subjected to various surface treatments and can be mechanically modified in terms of size and shape, providing customization based on specific dental applications.

The DRC composition is enriched with supplementary compounds like Fluoride-Releasing Resins, Quaternary Ammonium Compounds, and Bioactive Glass, adding to its versatility and expanding its potential applications and benefits.

Some embodiments of the present disclosure provide the incorporation of Fluoride-Releasing Resins into the DRC composition. These resins are advantageous due to their ability to offer sustained fluoride release. This feature has potential implications in aiding the remineralization of adjacent tooth structures, further providing a degree of anticariogenic protection.

Some embodiments of the present disclosure provide the integration of Quaternary Ammonium Compounds within the DRC composition. These compounds are renowned for their antimicrobial properties. The inclusion of these compounds can reduce or prevent bacterial colonization on the substrate's surface, which is a common concern in dental applications.

Some embodiments of the present disclosure provide the addition of Bioactive Glass. Bioactive Glass is known for its capability to form a bond with both hard and soft tissues. This bonding facilitates the regeneration and repair of tissues.

In some embodiments, the resin mixture, containing specific monomers, acts as the binding medium for the diamond particles, facilitating a cohesive structure in the finalized composite. In particular embodiments, the mixture incorporates between about 5-70 wt % of bisphenol A diglycidyl ether dimethacrylate (Bis-GMA), Urethane Dimethacrylate (UDMA), Triethylene Glycol Dimethacrylate (TEGDMA), Hydroxyethyl Methacrylate (HEMA), or Ethoxylated Bisphenol A Dimethacrylate (Bis-EMA). Such concentration ranges have been determined optimal for achieving a harmonious balance of mechanical properties within the composite.

Bisphenol A Diglycidyl Ether Dimethacrylate (Bis-GMA) is a primary resin monomer in dental composite resins, Bis-GMA is employed for its superior aesthetic and mechanical properties. Its concentration ranges from 10% to 60% by weight, with its viscosity often moderated by co-monomers such as triethylene glycol dimethacrylate (TEGDMA) or ethylene glycol dimethacrylate (EGDMA).

Urethane Dimethacrylate (UDMA) is another dimethacrylate monomer, UDMA exhibits a viscosity lower than Bis-GMA. It finds usage in dental composites, contributing both mechanical strength and ease of application, with concentrations ranging from 10% to 40%.

Triethylene Glycol Dimethacrylate (TEGDMA) is employed as a diluent to decrease the viscosity of Bis-GMA-based dental composites. Typically present in concentrations between 10% and 40%, TEGDMA complements the robust matrix provided by Bis-GMA. Its reduced viscosity improves the resin blend's workability while enhancing the mechanical strength and longevity of the polymerized material through cross-linking.

Hydroxyethyl Methacrylate (HEMA) is used in select dental bonding agents and composite resins, HEMA, a hydrophilic monomer, aids in bonding. Its presence, between 5% and 15%, may heighten susceptibility to water sorption.

Ethoxylated Bisphenol A Dimethacrylate (Bis-EMA, especially Bis-EMA (4)) is used an alternative or adjunct to Bis-GMA, Bis-EMA displays reduced viscosity due to the lack of hydroxyl groups inherent in Bis-GMA. Its concentration spans from 10% to 50%, and the monomer presents lower water sorption properties.

In one embodiment, the resin mixture may be enhanced by the addition of about 5-25 wt % ethylene glycol dimethacrylate. This component is added to augment the mechanical and adhesive properties of the composite. Ethylene glycol dimethacrylate serves as a cross-linking agent in dental restorative formulations. Its role in the matrix aids in optimizing the final characteristics of the restoration. While EGDMA is notable for its efficacy, a range of other dimethacrylate compounds and associated substances may serve a similar purpose in dental composite formulations.

In one embodiment, Triethylene glycol dimethacrylate (TEGDMA) is incorporated into dental restorative formulas. It's recognized for both its ability to reduce resin matrix viscosity and its cross-linking capacity.

Another embodiment utilizes Urethane dimethacrylate (UDMA). Though primarily a foundational resin, UDMA's multifunctional attributes make it conducive to cross-linking, further enhancing the composite's properties.

An alternative to the conventional Bis-GMA is the Bisphenol A ethoxylate dimethacrylate (Bis-EMA). Its structural absence of hydroxyl groups renders it less viscous, making it advantageous in certain formulations.

In other embodiments, 1,6-Hexanediol dimethacrylate (HDDMA) and 1,12-Dodecanediol dimethacrylate (DDDMA) might be employed as cross-linking agents, with the latter being recognized for its elongated chain structure.

In one embodiment, Trimethylolpropane trimethacrylate (TMPTMA) introduces a trifunctional monomer into the mix, potentially offering even more pronounced cross-linking capabilities.

While methacrylates dominate in dental applications due to their superior biocompatibility, some formulations may benefit from the inclusion of di- and tri-functional acrylates as cross-linking agents.

In one embodiment, Bisphenol A diglycidyl ether dimethacrylate (Bis-GMA), typically serving as a primary resin, can also contribute to cross-linking given its bifunctional characteristics.

It will be appreciated that the specific embodiments described herein are illustrative only and not intended to be limiting. Variations and modifications to the disclosed embodiments can be made and may be envisioned by those skilled in the art, without departing from the scope and spirit of the invention.

An essential feature of the DRC lies in its photopolymerization capability, achieved through the incorporation of photoinitiators. Photoinitiators are essential components in the polymerization process of dental resin materials. Camphorquinone (CQ) is used as photoinitiator for light-cured dental composites. Upon exposure to specific visible light, for example in the blue spectrum at approximately 468 nm, CQ absorbs this energy and transitions to an excited state. Subsequently, it collaborates with co-initiators, typically tertiary amines, generating free radicals. These radicals spearhead the polymerization of resin monomers, transforming the soft composite into a rigid and enduring structure.

Additionally, in one embodiment, both 9-(2,4,4,6-trimethylbenzoyl)-9-oxytho-9-phospha-fluoren (TMBOPF) and/or 9-(p-toluyl)-9-oxytho-9-phosphafuluorene (TOPF) are used as photoinitiators in dental composite formulations. They operate on a foundational principle analogous to CQ, creating free radicals when exposed to light, which then instigate the polymerization process. Integrating different photoinitiators or their combinations allows the modulation of the polymerization characteristics. Such variations can enhance attributes like the depth of cure, polymerization velocity, or the color constancy of the finalized dental restoration.

In certain embodiments, the DRC incorporates between about 0.01-0.5 wt % of specific photoinitiators like camphorquinone (CQ), 9-(2,4,4,6-trimethylbenzoyl)-9-oxytho-9-phospha-fluoren (TMBOPF), or 9-(p-toluyl)-9-oxytho-9-phosphafuluorene (TOPF). These photoinitiators are selected for their efficiency in initiating the polymerization process when exposed to a curing light.

In certain embodiments, Phenylpropanedione (PPD) when conjoined with CQ, boosts polymerization efficiency.

In certain embodiments, Lucirin TPO (LAP) independently generates radicals when subjected to UV light, enhancing the cure process.

In certain embodiments, Ivocerin (BAPO) demonstrates promising rapid polymerization kinetics, distinguishing itself in the field.

Some embodiments of the present disclosure provide formulations and compositions of dental resin composites (DRCs) optimized for polymerization and tailored for dental restoration applications. Some embodiments of the present disclosure address the need for efficient and effective photopolymerization, ensuring optimal physical properties and longevity of dental restorations.

To complement the photoinitiator, the dental resin composite (DRC) fundamentally includes a polymerizable resin matrix, a photoinitiator, and a co-initiator. The co-initiator serves a pivotal role in the polymerization process. These coinitiators work in tandem with the photoinitiators, ensuring a complete and robust polymerization of the composite.

In some embodiments, the co-initiator incorporated within the dental resin composite is present in specific concentration bounds, specifically ranging from about 0.01 to 0.5 wt %. This weight percentage is determined to ensure optimal polymerization.

In some embodiments, ethyl N,N-dimethyl-4-aminobenzoate (EDMAB) serves this role. EDMAB, when incorporated as a co-initiator, synergizes with the photoinitiator to produce radicals upon light exposure. These radicals, in turn, initiate and drive the polymerization of the resin matrix, converting it from a moldable material to a rigid dental structure.

An alternative embodiment employs 1,3-Diethyl-2-thiobarbituric acid as the co-initiator. While its operational principal parallels that of EDMAB in terms of radical generation and polymerization initiation.

In certain formulations, the dental resin composite can comprise one or more additional co-initiators, broadening the spectrum of radical generation and fine-tuning polymerization properties. Such co-initiators can be selected from a group which includes Ethyl 4-(dimethylamino)benzoate (EDAB), N,N-dimethyl-p-toluidine (DMPT), N,N-dihydroxyethyl-p-toluidine (DHEPT), Triethanolamine (TEA), and Cyanoethyl methylaniline (CEMA).

Some embodiments of the present disclosure provide a unique formulation known as the Diamond Resin Composite or DRC. This composite is notably characterized by the presence of diamond particles, judiciously selected to be present in a concentration of 30-60 wt %. The significance of this specific concentration range lies in achieving a balance between mechanical robustness and workability of the composite.

These diamond particles are intimately dispersed within and embedded in a specially formulated multi-monomer resin mixture. This resin mixture not only serves as the binding matrix for the diamond particles but also ensures their uniform distribution, thus providing the composite with its distinct structural integrity and performance attributes.

Additionally, the DRC also incorporates components vital for its polymerization process, specifically a photoinitiator and a coinitiator. These elements play a pivotal role in the curing or setting of the composite upon exposure to appropriate stimuli.

In some embodiments of the disclosure, the diamond particles integrated into the DRC are of synthetic origin. The method of synthesis for these synthetic diamonds is not limited to a single technique. In particular, the diamond materials are meticulously prepared employing techniques including one or more of:
a. High-pressure high-temperature (HPHT) synthesis, a well-regarded method known for its ability to produce diamonds of significant size and purity;
b. Microwave plasma-assisted chemical vapor deposition (MPCVD), notable for its precision and control in diamond layer growth;
c. Hot-filament chemical vapor deposition (HFCVD), recognized for its versatility and ability to deposit diamond on various substrates; and
d. DC plasma enhanced chemical vapor deposition (DC-PECVD), an advanced technique that offers controlled deposition parameters for tailored diamond properties.

It should be noted that the selection of the synthesis method can influence the morphological, structural, and performance characteristics of the diamond particles, thereby subtly influencing the properties of the final DRC.

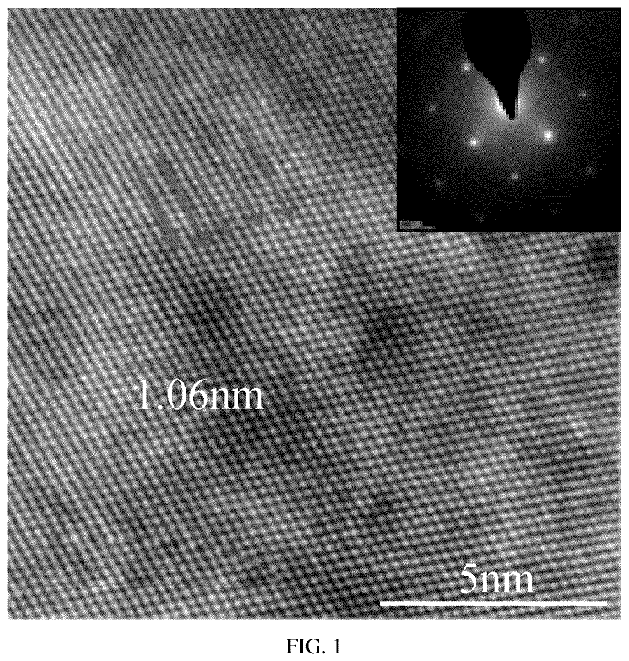

FIG. 1 shows an example of Transmission Electron Microscopy (TEM) micrograph showing a single crystalline diamond. The observed lattice structures represent the periodic arrangement of carbon atoms in the diamond crystal. In the TEM image, the clear atomic planes and the inherent crystallographic order of the diamond can be seen. The spacing between these lattice planes, along with the overall clarity and resolution of the image, attests to the high quality and single crystalline nature of the diamond sample.

A scale bar, prominently displayed at the bottom right of the micrograph, indicates a length of 5 nm. This scale bar serves as a reference, providing context to the size and spacing of the lattice structures observed in the diamond. Given this scale, it is evident that the visible lattice fringes are representative of the specific interplanar spacing of the carbon atoms in the diamond structure.

The Selected Area Electron Diffraction (SAED) pattern of the diamond sample was inserted on top right of the TEM image. The SAED pattern is a result of the interaction between the incident electron beam and the periodic crystal lattice of the diamond. The distinct diffraction spots arranged in concentric circles in the SAED pattern are indicative of the periodicity and symmetry of the diamond lattice. The relative positions, intensities, and distances between these diffraction spots provide critical information about the lattice constant, crystallographic orientations, and possible defects in the diamond.

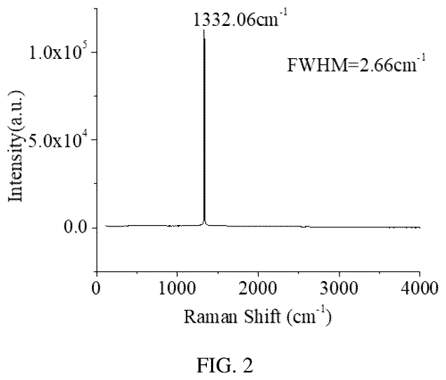

FIG. 2 displays an example of the Raman spectroscopy result for a synthetic diamond produced via the MPCVD process. The Raman spectrum was derived after efficiently removing the fluorescence background using Lorentz fitting techniques. A notable feature in the spectrum is the Raman characteristic peak situated at 1,332.12 cm⁻¹. Notably, no other characteristic peaks were observed beyond this value. This prominent Raman peak aligns closely with the standard Raman peaks of natural diamonds, which are typically found at 1,332 cm⁻¹. This alignment, combined with the lack of other peaks, emphasizes that the developed single crystal diamond sample doesn't contain non-diamond phases.

A minor deviation from the natural diamond's Raman peak was observed, marking a difference of 0.12 cm⁻¹. This shift suggests the presence of compressive stress within the diamond structure. Additionally, the full width at half maximum (FWHM) of the Raman peak was ascertained to be 2.69 cm⁻¹. Such a narrow FWHM distinctly indicates the high quality of the single crystal diamond. The limited width of this peak further supports the absence of lattice defects in the sample, testifying to its excellent crystalline quality.

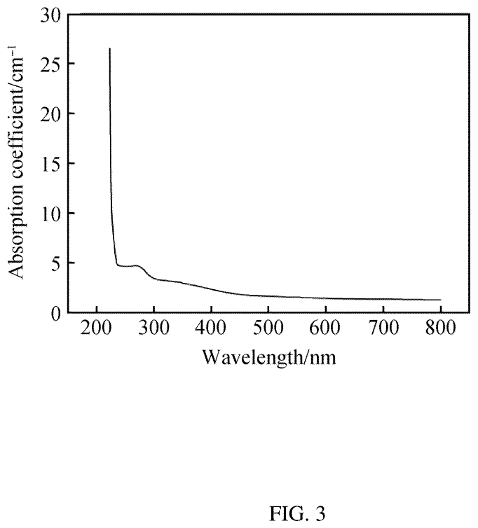

FIG. 3 shows an example of the UV-Vis spectrophotometer result of the MPCVD synthesized diamond. The absorption spectrum in the ultraviolet-visible band provides noteworthy insights into the sample's behavior.

Within the band interval of 500-800 nm, the sample's absorption coefficient remains relatively consistent, evidencing little variation. A shift is observed as one moves from the 500 nm region towards the blue and near-ultraviolet bands. As the incident wavelength diminishes, there is a monotonic increase in the absorption coefficient. This increase becomes especially prominent around the 270 nm range. This wider absorption band at approximately 270 nm can be attributed to the lone nitrogen center's formation. Such a center results from the introduction of nitrogen atoms during the sample's growth phase, thereby leading to this distinctive absorption peak.

Additionally, the optical absorption edge of the examined sample is pinpointed at the wavelength of 223 nm. This is near the 225 nm optical absorption edge commonly observed in natural type IIa diamonds, suggesting the synthetic diamond's alignment with its natural counterpart in this respect.

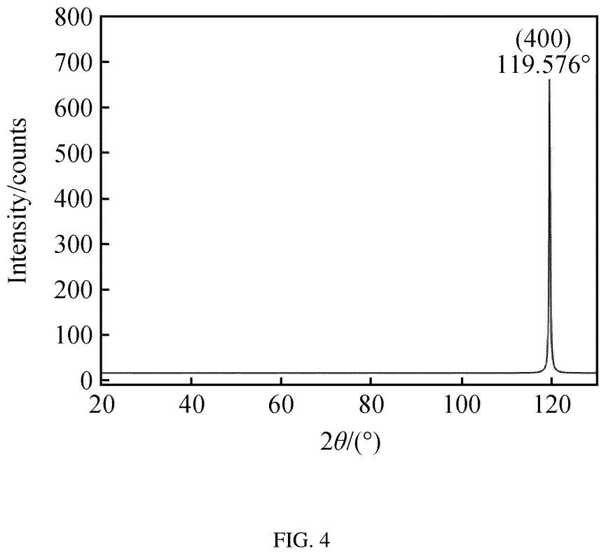

FIG. 4 shows an example of the XRD characterization results of the MPCVD synthesized diamond. The X-ray diffraction pattern of diamond samples shows that there is only one sharp diffraction peak in the range of 20°˜130°, and its position is at 2θ=119.576° (d=0.089 nm), corresponding to the crystal orientation of diamond (400), for Cubic structure with good crystallinity.

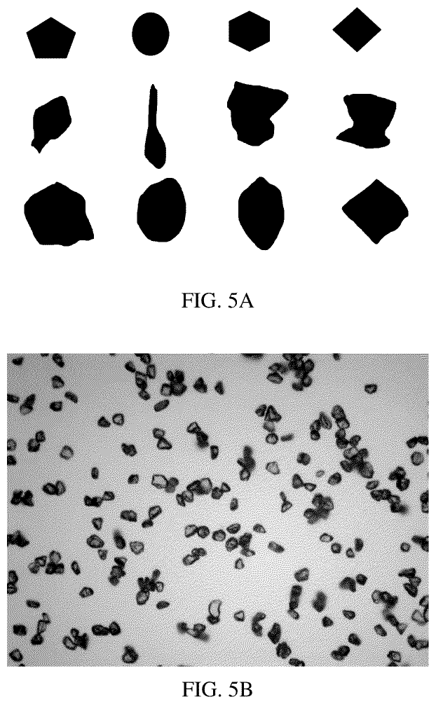

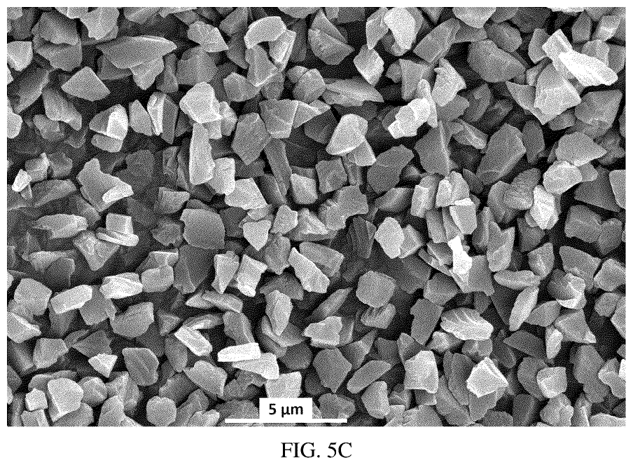

FIG. 5A shows an example of various shapes of synthetic diamond particles, each tailored for use in DRC and exhibiting distinct morphologies. Depending on the techniques used for size reduction, both classic geometric forms and irregular particle shapes can emerge. Such non-uniform configurations might arise from external influences like mechanical processing or unique growth conditions divergent from standard synthetic diamond production methods. Following the size reduction process, a rigorous purification procedure using a potent acid mixture is employed. This method facilitates the removal of impurities, specifically metal and non-diamond carbon contaminants, introduced during the milling stage.

FIG. 5B shows an optical microscope image of diamond particles after ultrasonic cleaning of the mechanical milling step. The diamond particles are relatively evenly distributed in the optical field. FIG. 5C shows a scanning electron microscope (SEM) image of micrometer size diamond particles after chemical cleaning. The diamond particle shows an average size of 1 micrometer. The particles are not agglomerated which allows further surface treatment such as doping, plasma treatment as described in this patent.

The mechanical milling of bulk diamond to produce particles utilizes a ball mill, which is a cylindrical device equipped for grinding or mixing materials. High-purity bulk synthetic diamond is introduced as the starting material, with the choice of hard milling media, such as tungsten carbide or steel, playing a pivotal role in determining the efficiency of size reduction. As the ball mill operates, the interplay of the balls energetically grinds and fractures the diamond, continuously reducing its size. Critical parameters, including the ball-to-diamond weight ratio, rotation speed, and mill chamber temperature, are optimized to achieve the desired nanoparticle size range efficiently. Intermittent sampling and monitoring, using methods like electron microscopy, ensure that the milling process is on the desired trajectory.

Post-milling, the diamond particles are separated from the milling media and potential impurities through methods like sieving or centrifugation. Subsequent cleaning procedures, typically employing solvents, remove any lingering contaminants. The finalized diamond particles are then analyzed using advanced techniques, such as Transmission Electron Microscopy (TEM), to ascertain their size distribution. Throughout the milling process, special consideration is given to potential heat generation and induced defects in the diamond structure. The optimization of conditions, including cooling mechanisms, if necessary, ensures the integrity and quality of the resultant particles.

Some embodiments of the present disclosure provide synthesis of diamond materials through various methods including high-pressure high-temperature (HPHT) synthesis and three chemical vapor deposition (CVD) techniques. Each method and its specific parameters, conditions, and results are further described herein.

The High-Pressure High-Temperature (HPHT) Synthesis is a method 27sed in some embodiments. Under extreme conditions, HPHT synthesis transforms carbon sources into synthetic diamond crystals. These synthetic diamonds can be differentiated by their elemental impurities. For instance, certain synthetic diamonds prepared through the HPHT method comprise nickel as an impurity with concentrations up to 200 parts per million (ppm). Others contain nitrogen not exceeding 100 ppm, while some have iron impurities up to 200 ppm. Additionally, cobalt impurities in synthetic diamonds produced by this method can reach concentrations up to 200 ppm, and magnesium may be present up to 100 ppm.

Another technique highlighted is the Microwave Plasma-Assisted Chemical Vapor Deposition (MPCVD). Diamonds produced via the MPCVD method can be characterized not only by their elemental composition but also by their production conditions. Some diamonds have nitrogen impurities with concentrations that do not exceed 200 ppm. Others may contain boron or silicon, with concentrations not exceeding 300 ppm and 1 part per thousand (ppt) respectively. In terms of the gas mixture introduced into the deposition chamber during the MPCVD process, it includes a carbon source like methane (CH4) or carbon dioxide (CO2), combined with hydrogen (H2) in an amount no less than 70 vol. % of the mixture. When it comes to growth conditions for the MPCVD method, diamonds may be synthesized at temperatures anywhere from 300 degrees Celsius up to 1400 degrees Celsius. The pressures during these processes can range between 1×10\hat{ }−7 Torr and 100 Torr.

In the realm of Hot-filament Chemical Vapor Deposition (HFCVD), the process typically employs a gas mixture comprised of carbon sources such as methane (CH4), carbon dioxide (CO2), or carbon monoxide (CO). Hydrogen (H2) is also included with its volume percentage in the gas mixture being no less than 80 vol. %. The HFCVD method involves heating a filament to temperatures ranging from 1000 degrees Celsius up to 2400 degrees Celsius. Pressure conditions during this process can vary, ranging anywhere from 1×10\hat{ }−4 Torr up to 760 Torr.

The DC Plasma Enhanced Chemical Vapor Deposition (DC-PECVD) method synthesizes diamonds under specific conditions and parameters. The gas mixture used consist of methane (CH4), carbon dioxide (CO2), carbon monoxide (CO), or acetylene (C2H2). Hydrogen is also typically added with its volume percentage in the mixture being no less than 50 vol. %. Growth temperatures for the DC-PECVD process can span between 900 degrees Celsius and 1500 degrees Celsius. The pressures applied during this procedure can range from 1×10\hat{ }−3 Torr to 760 Torr.

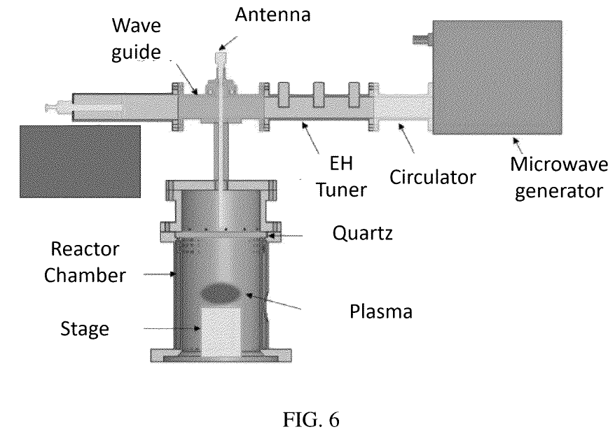

FIG. 6 is an example of a microwave plasma assisted chemical vapor deposition (MPCVD) device tailored for the efficient synthesis of high-quality diamond materials. The diamond materials will mechanically break down to particles for use in DRC. This equipment seamlessly integrates multiple systems, ensuring the optimal conditions for diamond growth are consistently achieved.

The gas delivery system of the MPCVD device ensures a consistent and controlled supply of essential gases into the apparatus. This system adeptly handles the introduction of a carbon source gas, hydrogen gas, and/or nitrogen gas. These gases can be introduced individually or in a pre-determined mixture, depending on the specific requirements of the diamond synthesis. Incorporated flow controllers and regulators maintain the desired flow rates and pressures, setting the stage for the necessary chemical reactions to take place.

Shown in FIG. 6, central to the MPCVD device is the reactor chamber, a meticulously designed environment where diamond synthesis takes place. Constructed from materials that can endure elevated temperatures and pressures, this chamber safely facilitates the diamond growth process. Within its confines, a substrate is positioned, serving as the foundational layer upon which the diamond film is formed.

Providing the necessary energy for generating plasma, an energy source, compatible with microwave frequencies, is integrated into the design. It ensures that the gases introduced into the reactor chamber are excited to a plasma state, which is fundamental for diamond synthesis in this method.

Another essential component of the MPCVD system is the vacuum system. This vacuum system ensures that the reactor chamber maintains a near-perfect vacuum environment to generate plasma and later growth of diamond. This absence of extraneous gases and particles ensures that the conditions within the chamber are conducive for the formation of pure and high-quality diamond films.

Once the diamond synthesis process concludes, by-products and unreacted gases need to be safely and efficiently removed from the reactor chamber. This task is accomplished by the exhaust system, which channels these gases away from the chamber and ensures the equipment's safety and longevity. The formation of the diamond film capitalizes on the active groups and abundant hydrogen atoms present in the plasma, resulting in a product of superior quality.

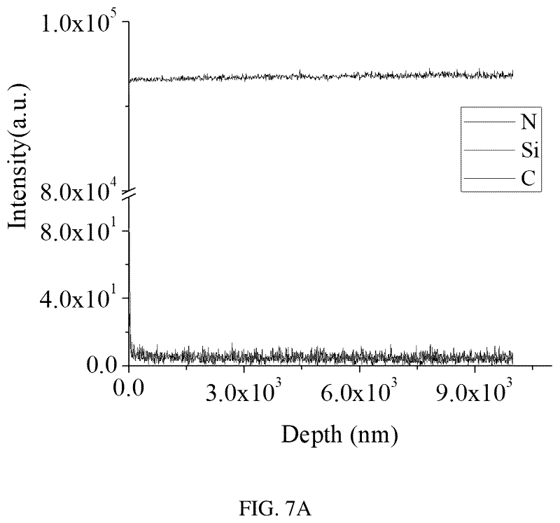

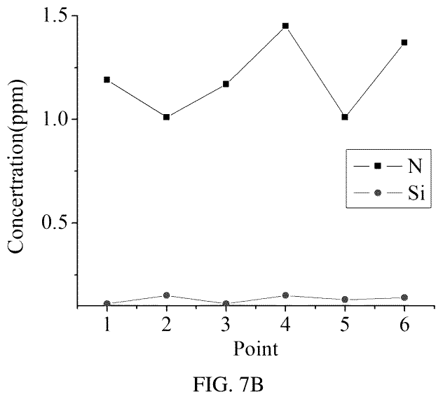

In FIG. 7A, the variation in secondary ionic strength across different depths is illustrated using the Secondary Ion Mass Spectrometer (SIMS) for the synthetic diamond sample produced through the MPCVD process. To evaluate impurity levels in the sample, concentrations of Nitrogen and Silicon were methodically analyzed at six distinct test points, reaching a maximum depth of 10 μm. As depicted in FIG. 7B, the data reveals that the sample contains minimal amounts of nitrogen and silicon atoms. Specifically, the average atomic concentrations for nitrogen and silicon were found to be 1.82×10\hat{ }(−6) and 0.15×10\hat{ }(−6), respectively. These findings highlight the outstanding purity of the synthesized diamond sample.

Some embodiments of the present disclosure provide a method for forming a doped region within a diamond substrate. The innovative process leverages the precision of repeated cycles of doping and annealing to achieve a controlled doping profile in the substrate.

Initially, the surface of the diamond substrate is doped with selected dopants. Post doping, an annealing process is executed on the diamond substrate to drive the dopants into the substrate. This drive-in method enables the penetration of dopants to a certain depth within the substrate. For a more controlled and refined doping profile, this doping and annealing process is iteratively repeated. This repetition offers a method to precisely control the dopant's concentration profile throughout the doped region of the diamond substrate.

In specific embodiments, diamond particles that are doped may optionally comprise a fluorine element. The incorporation of fluorine can be achieved through various means. One technique introduces fluorine-containing reactive gas precursor molecules into the diamond's chemical vapor deposition (CVD) synthesis process. Another approach incorporates fluorine into the diamond substrate through ion implantation. Following this implantation, a subsequent heat treatment is administered to further drive and embed the fluorine dopants within the substrate.

Beyond the doping aspects, further characteristics of the diamond particles are highlighted. For instance, specific embodiments of these diamond particles exhibit a size dimension ranging between 10 nm to 100 μm. This range offers flexibility and adaptability for different applications where the size of the diamond particle is crucial. The diamond particles, in certain embodiments, possess a non-spherical shape. Their unique shape is quantified by a sphericity metric that varies between 0.07 and 0.97. Such diverse shapes can have implications on properties like light scattering, surface area, and interlocking characteristics when the particles are utilized in composite materials.

Before integrating these diamond particles into diamond resin composite (DRC), surface treatment can be performed. Some embodiments have the diamond particles undergo a plasma surface treatment. This treatment is anticipated to enhance the interfacial interaction between the diamond particles and the surrounding matrix. However, in alternative embodiments, the diamond particles may remain untreated, preserving their original surface chemistry and attributes.

Within the field of diamond particle treatment, some embodiments of the present disclosure provide advancements in the surface treatment of diamond particles, specifically before their integration with other materials to form diamond resin composites (DRC). By pre-treating these diamond particles, a noticeable enhancement in the overall characteristics of the composite can be achieved, primarily due to the modulated interactions between the diamond particles and the matrix.

Certain diamond particles undergo plasma treatment prior to their incorporation into the DRC. Such a treatment bestows upon the particles unique surface characteristics, which can enhance interfacial adhesion within the composite. However, it should be noted that the treatment is not universal, and some particles might be utilized in their native, untreated state.

One of the specific treatment approaches involves hydrogen termination of the diamond particles' surface. This involves replacing extant surface groups with hydrogen atoms, imparting specific chemical and electronic properties to the particles. This method of termination is distinct in its ability to modify the surface properties.

Another approach to surface treatment includes oxygen termination of the diamond particles. By oxygen-terminating the particles, their oxidizing potential can be augmented. As with the previous methods, not all diamond particles are necessarily subjected to this specific termination.

In yet another variant, diamond particles can be treated to achieve fluorine termination. The infusion of fluorine groups on the particle surface can significantly alter their surface reactivity and hydrophobicity. This makes them suitable for certain specialized applications or to enhance interactions within the DRC. As with other terminations, this is not a universal treatment, and some diamond particles might be used in their original, untreated form.

Collectively, these advanced treatment methods and terminations are expected to play a crucial role in tailoring the properties of diamond resin composites and other domains where treated diamond particles find application.

The present disclosure elucidates a meticulously crafted diamond resin composite (DRC). At the heart of this formulation are synthetic diamond particles, which are embedded seamlessly within a multi-monomer resin mixture. This integration ensures an equitably balanced dispersion of the diamond phase throughout the resin matrix. The concentration of these diamond particles is judiciously maintained between 30-60 wt %, highlighting their integral role within the composite.

This resin mixture, beyond serving as a matrix for the diamond particles, comprises a distinct component-bisphenol A diglycidyl ether dimethacrylate or its close variant, Bisphenol A-glycidyl methacrylate. These constituents occupy between 30-40 wt % of the composite. Their inclusion, at such specified concentrations, is pivotal in bequeathing the composite with its desired mechanical and chemical attributes.

A quintessential element of the DRC's formation is the process of photoinitiation, which is indispensable for the polymerization of the composite. This process is steered by the presence of specific agents, such as camphorquinone (CQ), 9-(2,4,4,6-trimethylbenzoyl)-9-oxytho-9-phospha-fluoren (TMBOPF), or 9-(p-toluyl)-9-oxytho-9-phosphafuluorene (TOPF). These agents are carefully blended into the composite in concentrations ranging from 0.01-0.3 wt %. Depending on the curing conditions or the end application, the choice of photoinitiator can be adeptly adjusted.

In concert with the photoinitiators, coinitiators play an instrumental role in achieving an effective polymerization. Ethyl N,N-dimethyl-4-aminobenzoate (EDMAB) or 1,3-Diethyl-2-thiobarbituric acid are introduced within the composite, each falling within the concentration bandwidth of 0.01-0.3 wt %. The cooperative action between the photoinitiators and the coinitiators ensures swift and uniform polymerization when the composite is subjected to a pertinent light source.

Expanding the composite's functionality, the DRC can be optionally augmented with a medley of constituents. Some embodiments of the present disclosure provide components in diamond resin composites designed to enhance the physical, mechanical, and antibacterial properties of the resulting restorations. These components are introduced in specified weight percentages to achieve desired effects and optimal performance in various dental applications. In the following sections, each of the components and their significance in diamond resin composites is elaborated as follows.

Fluoride-Releasing Resins, when present in a weight range of approximately 1% to 20%, play a pivotal role in dental restorative compositions. The resins continuously release fluoride ions over time. The released fluoride ions contribute to the remineralization of tooth structure, especially in the areas adjacent to the restoration. Such a mechanism serves as a preventative measure against dental caries, mitigating the risk of recurrent caries near the restoration. Further, these resins provide an added layer of protection, reinforcing the tooth's natural defense mechanisms against acidic attacks and bacterial invasion.

The integration of Quaternary Ammonium Compounds (QACs), preferably within the range of 0.5% to 5% by weight, infuses the dental composite with substantial antibacterial properties. QACs operate by disrupting bacterial cell membranes, effectively inhibiting bacterial proliferation on and around the restorative material. This action curtails plaque accumulation, promoting a healthier oral environment, and enhances the longevity of dental restoration.

Silver nanoparticles, when incorporated in amounts ranging from 0.05% to 2% by weight, impart a distinct antimicrobial attribute to the dental resin composites. These nanoparticles function by interfering with the bacterial DNA and obstructing the respiratory chain enzymes, preventing bacterial colonization on the composite's surface. The ultimate result is a reduced propensity for biofilm formation, further preventing dental caries initiation.

Zinc Oxide Nanoparticles are introduced in dental composites in proportions ranging from 1% to 10% by weight. Not only do they exhibit antibacterial qualities, but they also possess UV-blocking characteristics. The dual action of these nanoparticles—antibacterial and UV resistance—enhances the resin composite's defense against bacterial growth and discoloration upon exposure to ultraviolet light, respectively.

Chlorhexidine, present in a range of 0.5% to 5% by weight, is renowned for its profound antibacterial capabilities. Within the dental composite matrix, it offers sustained antibacterial effects. This component actively reduces bacterial colonization, ensuring that restorations remain less susceptible to secondary caries. Its long-lasting effects provide a safeguard that prolongs the restoration's functional life while maintaining the health of the surrounding oral tissues.

In dental resin composites, Bioactive Glass, incorporated in quantities ranging from 5% to 15% by weight, has multifaceted benefits. These biocompatible materials can form bonds with both hard and soft oral tissues. Their primary advantage lies in their ability to promote tooth remineralization, while also minimizing tooth sensitivity post-restoration. The presence of Bioactive Glass fosters better biological integration between the restoration and the adjoining tissue, ensuring both structural integrity and enhanced protective benefits to the tooth structure.

Central to the method is the process of photopolymerization. When the DRC, once applied, is illuminated by a specific wavelength of light, it undergoes a transformative phase transition. The composite hardens and stabilizes, resulting in the effective restoration of the dental surface in question.

In terms of granularity, the DRC isn't just a monolithic application. Its efficacy is discerned when directly instilled into cavities, whether they are in the anterior or posterior teeth. Customization is a key attribute of the DRC. It can be deftly shaped to snugly fit within dental cavities, subsequently being cemented to function as inlays and onlays. Not just restricted to structural restoration, the DRC also finds a revered place in the realm of cosmetic dentistry. Minor imperfections and blemishes can be seamlessly addressed through dental bonding.

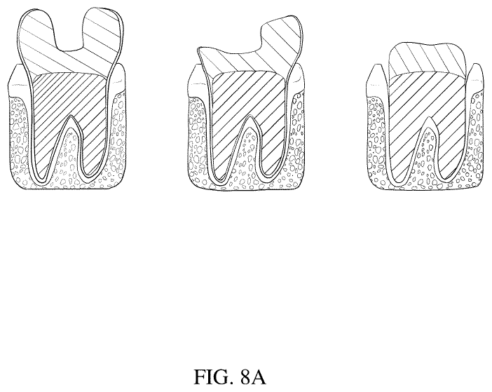

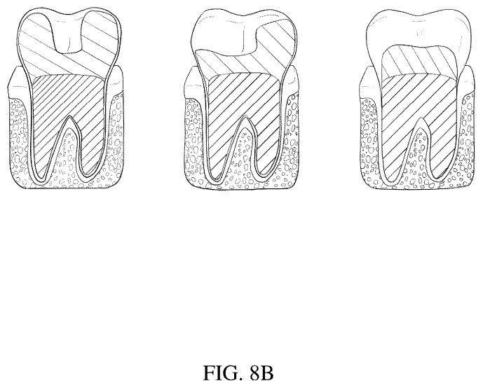

FIG. 8B depict a tooth subjected to restoration using diamond resin composite (DRC). An inlay typically addresses tooth structure losses within the cusps or biting surface. The precision-crafted DRC inlay fits snugly into a prepared cavity, replicating the intricacies of natural tooth contours. Seamlessly integrated with the extant tooth structure, this inlay not only promises aesthetic enhancement but also offers durability. The choice of DRC in this restoration ensures minimal wear against the opposing teeth and mirrors the coefficient of thermal expansion of a natural tooth, positioning it as an ideal material for such interventions.

Central to FIG. 8B is an onlay restoration, where the application extends over one or more of the tooth's cusps. The onlay, constructed from DRC, serves to shield and rejuvenate areas of the tooth compromised by damage or weakness. The reinforced nature of DRC bestows upon the onlay significant resilience, ensuring its capability to resist masticatory pressures. Moreover, the inherent bonding attributes of DRC ensure a strong amalgamation with the underlying tooth structure, drastically reducing risks associated with debonding or potential leakage.

The rightmost section of FIG. 8B depicts a crown restoration using DRC. Crowns, as portrayed, are structures enveloping the tooth's entirety above the gum line. The meticulously designed DRC crown echoes the natural tooth's morphology, shade, and translucency. Using DRC in crown restorations offers several advantages, including impressive fracture resistance, anti-chipping properties, and adeptness at distributing occlusal forces uniformly. These technical benefits, coupled with the composite's innate optical characteristics, culminate in a restoration that balances functional reliability with aesthetic finesse.

In a more preventive capacity, the DRC is applied as sealants, specifically targeting the occlusal surfaces of molars and premolars to prevent potential decay. The occlusal surface, characterized by deep fissures and grooves, often becomes a harbinger for bacterial colonies and potential decay. The DRC, in its flowable form, is exhibited in the visuals adapting meticulously to these grooves, offering comprehensive coverage that traditional sealants may not achieve with the same precision.

Post-application, the molar's occlusal surface is encapsulated in a translucent layer of DRC, establishing a formidable defense against cariogenic agents. This barrier, formed due to the DRC's inherent bonding and wear-resistant properties, not only thwarts bacterial infiltration and subsequent acid attacks but also promises prolonged durability, reducing the frequency of reapplications. Thus, the innovative use of DRC as portrayed in the illustrations promises an elevated standard of preventive dental care, especially for the intricate occlusal surfaces.

The adhesive strength of diamond resin composite is particularly valuable in orthodontics. Brackets, fundamental to orthodontic treatments, can be securely bonded to teeth using the DRC. The composite's remedial capacity is further highlighted in root surface caries restorations, wherein it becomes instrumental in counteracting decay on the tooth's root surfaces.

the DRC can also be applied in Pediatric. Primary teeth, which might be marred by cavities, fractures, or certain congenital conditions, can be treated, and restored using the DRC. The composite's adaptability is manifested when it addresses diastema, serving as a bridge to seal gaps between teeth, or when it comes to the rescue of teeth subjected to bruxism or acid erosion.

The illustrations demonstrate the use of diamond resin composite (DRC) in advanced dental restorations. The DRC is employed for posts and cores in teeth that have undergone root canal treatments. This application leverages the bonding and strength properties of the DRC, providing a foundation for subsequent prosthetic work and enhancing the durability of endodontically treated teeth.

The DRC is also utilized in Class IV and V restorations. The depicted Class IV restoration is applied to incisors or canines that have sustained fractures or chips. The DRC's capability to match the appearance of the natural tooth is evident in the restoration, ensuring continuity in both structure and aesthetics. The Class V restoration focuses on areas of teeth with decay, wear, or abrasions near the gum line. Here, the DRC serves as a protective layer against further decay while restoring the tooth's structural and visual integrity. The expansive purview of the DRC spans the gamut of dental prosthetics. From mending minor imperfections such as chips on denture teeth to the creation of holistic solutions such as complete dentures for edentulous patients, the DRC is indispensable. It aids in the formation of dental crowns, designed to resurrect damaged teeth, and assists in the installation of dental bridges, effectively replacing missing teeth.

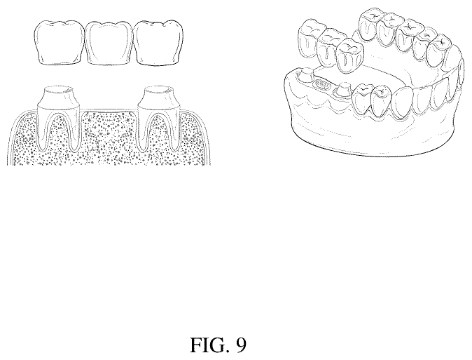

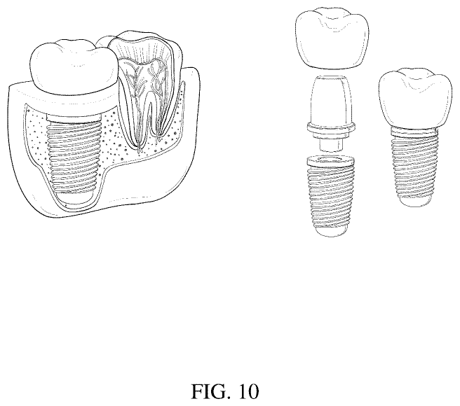

FIG. 9 provides another application example of DRC. Dental bridges, including resin-bonded bridges (often termed “Maryland bridges”), are prosthetics designed to replace missing teeth. The procedure begins by minimally preparing the teeth adjacent to the gap, followed by taking impressions of the custom-made bridge. The bridge features a replacement tooth attached to wings, typically made of metal or porcelain. These wings are designed to bond onto the backside of the adjacent teeth using a composite resin.

Maintaining oral hygiene and regular dental check-ups are crucial for the bridge's longevity. FIG. 9 shows an example of using DRC for dental bridge. For attachment, the internal surfaces of the wings are roughened to optimize bonding. After applying a bonding agent to the prepared teeth, the diamond resin composite is used to bond the bridge in place. The resin is then cured using a special light. After bonding, any excess material is removed, and the bridge is polished for a smooth finish.

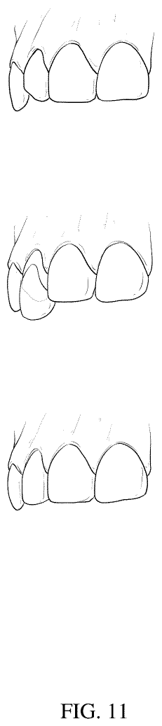

FIG. 10 shows an example of using DRC for dental implant. The present disclosure relates to the application and utilization of resin composite materials in conjunction with dental implant procedures. Dental implants, as known in the art, are typically fabricated from biocompatible materials such as titanium or zirconia and serve as foundational structures for supporting dental prostheses.

In one embodiment, during the osseointegration phase of the dental implant, a protective covering, specifically a temporary crown, is employed. The crown is comprised of a diamond resin composite material, functioning not only to protect the implant's abutment but also to serve as an interim placeholder. This resin-based crown ensures the abutment remains undisturbed and uncontaminated during the healing and integration process.

Some embodiments of the present disclosure provide the use of diamond resin composite for fabricating provisional crowns preceding the affixation of the permanent prosthesis. These provisional structures facilitate preliminary assessments encompassing aesthetics, functional dynamics, and biological tissue responses. Furthermore, when the final prosthesis, potentially crafted from ceramic materials like porcelain, is prepared for placement, a resin-based cement is utilized. This cement, composed of resin composite, is applied to bond the prosthesis securely to the implant's abutment, ensuring a durable and lasting connection.

Veneers, often sought for their aesthetic appeal, can be crafted from the DRC. These are thin protective layers, almost akin to shields, that are firmly bonded to the anterior portion of teeth, giving them a rejuvenated appearance. In instances where a tooth's core is compromised, and there's a looming crown placement, the DRC serves as the foundation for core build-ups. Its inherent strength is also tapped into for splinting, especially when certain teeth exhibit instability. By connecting these unstable teeth to their stable counterparts, stability is reinstated. Furthermore, teeth that might be teetering on the edge of vulnerability can be fortified using the DRC, thereby enhancing their resilience. In the sequence of illustrations provided, the transformation of a tooth through the application of a diamond resin composite (DRC) veneer is comprehensively depicted. FIG. 11 depicts this transformative process.

The topmost figure portrays a sample tooth prior to veneer restoration. The tooth's surface may show discolorations, imperfections, or slight structural anomalies, all indicative of the typical reasons one might seek a veneer restoration. This image sets the stage for the viewer, providing a baseline representation against which the subsequent transformations can be more profoundly appreciated.

The middle figure shows the process of placing the DRC veneer onto the tooth is detailed. Here, the veneer, fabricated from the diamond resin composite, is meticulously aligned with the prepared tooth surface. As captured in this representation, the DRC's adaptability and malleability are emphasized. The veneer's shape and contour, even at this interim stage, hint at its eventual seamless integration with the tooth. The unique bonding properties of the DRC, integral to ensuring a durable and resilient union between the veneer and the tooth, are evident in the intimate interface shown.

The final, lowermost figure shows the tooth post the DRC veneer restoration. With the veneer now fully set and polished, the tooth's appearance is dramatically enhanced. The DRC veneer's translucency, color consistency, and lifelike surface texture are vividly portrayed, reflecting its capacity to mimic the characteristics of natural teeth. The veneer not only addresses the tooth's initial imperfections but elevates its aesthetic appeal, demonstrating the dual functionality and cosmetic advantages of using DRC in dental restorations.

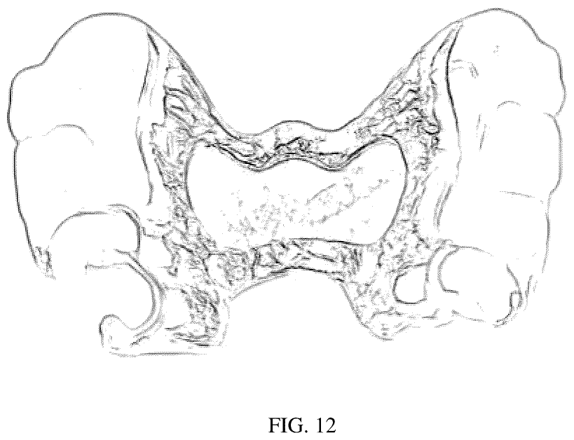

FIG. 12 is another example of an innovative application of diamond resin composite materials in the realm of dental prosthetics, more specifically for the fabrication and enhancement of combined maxillary prostheses.

A combined maxillary prosthesis is employed to rehabilitate patients with a partial or complete loss of maxillary teeth and structures. This prosthesis integrates with both remaining natural teeth and implants, ensuring a comprehensive restoration that mimics the functionality and aesthetics of the natural maxillary arch.

In one embodiment, the prosthetic framework is constructed using diamond resin composite, prized for its unique combination of strength, durability, and aesthetic appeal. The diamond particles incorporated into the resin matrix confer enhanced mechanical properties, providing resistance against wear, chipping, and fracture. The impressions or digital scans of the patient's maxillary arch serve as the foundational template for sculpting the prosthesis. Once the framework is devised, the remaining gaps are filled with meticulously crafted prosthetic teeth, also formed from diamond resin composite.

The diamond resin composite's inherent translucence enables the prosthesis to emulate the natural shimmer and vitality of dental enamel. Moreover, the material's versatility allows for a seamless integration with underlying implants, brackets, and retention elements. The bonding process, leveraging the compatibility between the diamond resin composite and adhesives, ensures that the prosthesis remains securely anchored, providing patients with a stable and comfortable bite.

Another advantage of the diamond resin composite is its adaptability, permitting chairside adjustments and modifications if needed. This adaptability ensures an impeccable fit and alignment of the prosthesis, preventing potential complications such as misalignment or occlusal discrepancies.

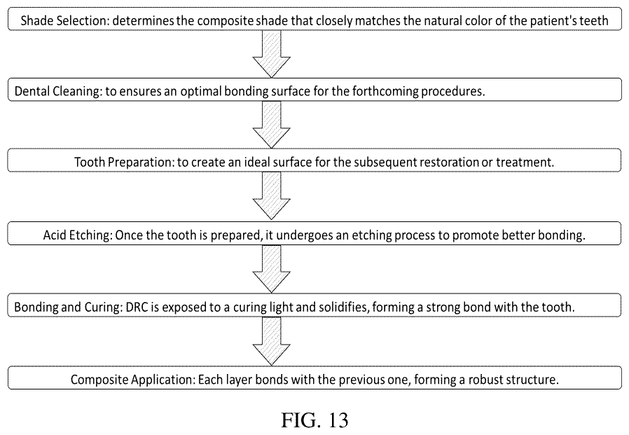

The subsequent discourse illuminates an innovative dental restoration approach predicated upon the use of diamond particles harmonized with a light-curable resin. This amalgamation forms the Diamond Resin Composite. Once prepared, this composite is applied to dental structures that exhibit signs of degradation or require restoration. FIG. 13 provides an illustrative example of the steps that can be followed for dental restoration using DRC.

In the dental restoration process using diamond resin composite, the initial and most pivotal steps involve the selection of the appropriate DRC shade. This is achieved by assessing the patient's natural tooth color using a commercially available shade guide under standardized lighting conditions. It's imperative that this selection be done prior to any tooth manipulation, ensuring that potential dehydration or discoloration effects don't compromise the accuracy of the shade match.

Following shade selection, the tooth's surface is meticulously cleaned. Dental professionals utilize prophylaxis paste with rotary dental tools or ultrasonic scalers to remove any superficial contaminants like plaque, calculus, or stains. Once cleaned, the tooth is thoroughly rinsed with water and air-dried using a dental syringe to achieve an uncontaminated and dry bonding surface.

Preparation of the tooth surface comes next. With the help of dental burs of varying shapes and grits, the dentist focuses on removing decayed tissue, remnants of old restorations, or any undercuts. The goal is to conserve as much of the natural tooth structure as possible while creating a surface that offers mechanical retention to the composite. Sharp edges and corners are meticulously rounded to reduce stress concentrations and ensure optimal bonding conditions.

Once the tooth is prepared, it undergoes an acid etching process to foster enhanced bonding of the DRC. A gel, commonly containing phosphoric acid in a concentration of around 30-40%, is uniformly applied to the prepared tooth surface. This application lasts for a brief period, typically varying based on whether enamel or dentin is being etched. Post etching, the tooth undergoes a comprehensive rinsing and is dried with care to leave the dentin slightly moist.

Following etching is a bonding step. A thin layer of a dental bonding agent is carefully brushed onto the etched surface. Once applied, any excess bonding agent is dispersed with gentle air. Subsequently, a dental curing light, which usually emits light in the 400-500 nm wavelength range, is utilized to polymerize the bonding agent for a duration that aligns with the guidelines.

In some embodiments, the restoration process culminates with the application of the DRC. The composite is incrementally layered into the tooth preparation. Dental professionals contour each composite layer, ensuring it resembles the natural topography of the tooth. Each of these layers is then set or cured using the dental curing light. This layering and curing process is reiterated until the tooth restoration achieves its desired form and function.

What is claimed is:

1. A diamond resin composite (DRC) comprising a multi-monomer resin mixture, diamond particles embedded within the multi-monomer resin mixture, a photoinitiator, and a coinitiator.

2. The diamond resin composite (DRC) of claim 1, wherein the diamond particles are made from bulk diamond, and wherein the bulk diamond is of synthetic origin.

3. The diamond resin composite (DRC) of claim 1, wherein the multi-monomer resin mixture comprises a dental diamond resin composite including a resin matrix, wherein the resin matrix includes one or more of: Bisphenol A-glycidyl methacrylate (Bis-GMA) in a concentration ranging from 10% to 60% by weight; Triethylene glycol dimethacrylate (TEGDMA) in a concentration ranging from 10% to 40% by weight; Urethane dimethacrylate (UDMA) in a concentration ranging from 10% to 40% by weight; Hydroxyethyl methacrylate (HEMA) in a concentration ranging from 5% to 15% by weight; and Ethoxylated Bisphenol A dimethacrylate (Bis-EMA) in a concentration ranging from 10% to 50% by weight.

4. The diamond resin composite (DRC) of claim 3, wherein the resin matrix comprises of Bis-GMA.

5. The diamond resin composite (DRC) of claim 3, wherein the resin matrix primarily comprises TEGDMA.

6. The diamond resin composite (DRC) of claim 3, wherein the resin matrix primarily comprises UDMA.

7. The diamond resin composite (DRC) of claim 3, wherein the resin matrix comprises HEMA.

8. The diamond resin composite (DRC) of claim 3, wherein the resin matrix comprises Bis-EMA.

9. The diamond resin composite (DRC) of claim 3, wherein the resin blend further comprises 5-25 wt % ethylene glycol dimethacrylate.

10. The dental resin composite (DRC) of claim 1, where the diamond resin composite comprises a photopolymerization capability and at least one photoinitiator wherein said photoinitiator initiates polymerization of resin monomers upon exposure to light.

11. The dental resin composite (DRC) of claim 10, wherein the photoinitiator comprises camphorquinone (CQ).

12. The dental resin composite (DRC) of claim 11, wherein upon exposure to visible light, said CQ absorbs said light energy and transitions to an excited state thereby inducing polymerization.

13. The dental resin composite (DRC) of claim 11, wherein the photoinitiator comprises at least one of 9-(2,4,4,6-trimethylbenzoyl)-9-oxytho-9-phospha-fluoren (TMBOPF) or 9-(p-toluyl)-9-oxytho-9-phosphafuluorene (TOPF).

14. The dental resin composite (DRC) of claim 11, wherein a concentration of the photoinitiator is between about 0.01-0.5 wt %.

15. The dental resin composite (DRC) of claim 11, wherein the photoinitiator comprises at least of camphorquinone (CQ), 9-(2,4,4,6-trimethylbenzoyl)-9-oxytho-9-phospha-fluoren (TMBOPF), and 9-(p-toluyl)-9-oxytho-9-phosphafuluorene (TOPF).

16. The dental resin composite (DRC) of claim 11, further comprising Phenylpropanedione (PPD) conjoined with CQ, wherein a combination amplifies polymerization efficiency.

17. The dental resin composite (DRC) of claim 11, further comprising Lucirin TPO (LAP), wherein said LAP augments the curing process.

18. The dental resin composite (DRC) of claim 11, further comprising Ivocerin (BAPO).

19. A method of preparing a dental restoration, comprising applying the dental resin composite (DRC) of claim 1 to a dental site, and exposing the DRC to light to induce polymerization, wherein the light has a wavelength of approximately 468 nm.

20. A diamond resin composite (DRC) comprising: diamond particles at a concentration of 1-60 wt % and of synthetic diamond origin; a multi-monomer resin mixture, wherein the diamond particles are embedded in the multi-monomer resin mixture,e and wherein the resin blend comprises 30-40 wt % bisphenol A diglycidyl ether dimethacrylate or Bisphenol A-glycidyl methacrylate.; 0.01-0.5 wt % camphorquinone (CQ), or 9-(2,4,4,6-trimethylbenzoyl)-9-oxytho-9-phospha-fluoren (TMBOPF) or 9-(p-toluyl)-9-oxytho-9-phosphafuluorene (TOPF) as the photoinitiator; 0.01-0.5 wt % ethyl N,N-dimethyl-4-aminobenzoate (EDMAB) or 1,3-Diethyl-2-thiobarbituric acid as the coinitiator.
